(19) United States
(12) Patent Application Publication
(10) Pub. No.: US 2009/0099515 A1
(43) Pub. Date: Apr. 16, 2009

(54) TRANS-CERVICAL CATHETER HAVING A CONICAL-SHAPED BALLOON

(75) Inventor: Wendy Quilter, South Jordan, UT (US)

Correspondence Address: KIRTON AND MCCONKIE, 60 EAST SOUTH TEMPLE,, SUITE 1800, SALT LAKE CITY, UT, 84111, US

(73) Assignee: UTAH MEDICAL PRODUCTS, INC., Salt Lake City, UT (US)

(21) Appl. No.: 11/870,491

(22) Filed: Oct. 11, 2007

(51) Int. Cl. A61M 25/10

(52) U.S. Cl. 604/96.01

(57) ABSTRACT

This application relates generally to a trans-cervical balloon catheter. The catheter may have an elongated body for insertion into the uterus. Located distally on the elongated body, the catheter may have a balloon that has a substantially conical-shaped proximal end when inflated. The balloon may comprise a proximal origin and a distal origin with a longitudinal midpoint located midway between the origins. The balloon may have a plane of maximum diameter located between the distal origin and the longitudinal midpoint. The proximal end of the balloon may taper from the plane of maximum diameter towards the proximal origin of the balloon in any manner that gives the proximal end of the balloon a conical-shaped appearance.

### BACKGROUND OF THE INVENTION

This application relates generally to a balloon for a trans-cervical catheter. More specifically, this application relates to a balloon with a substantially conical-shaped proximal end for use with a trans-cervical catheter. Additionally, this application relates to methods for making and using the described trans-cervical balloon catheter.

Currently, there are several diagnostic procedures that require entry into the uterus. Some examples of such procedures may include trans-vaginal ultrasound (“TVUS”) with saline infusion, also known as saline infusion sonography, and hysterosalpingography (“HSG”). The process of TVUS may involve inserting a fine flexible catheter into the cervical canal or the uterus and then injecting a sterile saline solution into the uterus. The solution may expand the uterus so that the uterus may be observed sonographically with an ultrasound scanner. The process of HSG is a radiographic method used for imaging anatomical structures of the uterus and fallopian tubes. Like TVUS, HSG may involve inserting a fine flexible catheter into the cervical canal or uterus and injecting a solution into the uterus. However, the solution in HSG is generally a contrast medium, such as an iodinated fluid. Once the contrast medium has been injected, radiography may then be performed to provide imaging information concerning the uterus and fallopian tubes.

In both of these procedures, the catheters used to deliver fluid to the uterus may have means for sealing off the uterus in order to prevent fluid backflow into the vaginal canal. Furthermore, the catheters may also have anchoring means to prevent the catheter from being dislodged during the procedure. In some instances, an inflatable balloon that is located near the distal tip of a catheter may act as both the sealing and anchoring means. Such a balloon is often made from an elastomeric material that allows the balloon to inflate and deflate. A catheter with such a balloon may be inserted into the cervical canal or uterus while the balloon is deflated. Once inserted and positioned, the balloon may be inflated and the catheter may be retracted to place the balloon against the internal orifice of the uterus or the wall of the cervical canal. After the diagnostic fluid has been injected and the visualization procedure performed, the balloon may be deflated and the catheter may be removed.

However, conventional balloons for trans-cervical catheters used in uterine imaging may have several problems associated with them. For example, some balloons may be designed to be inflated inside of the uterine cavity. One important disadvantage of such balloons is that they may sit above the internal orifice and may tend to block portions of the uterus during imaging. In this manner, such balloons may make it difficult or impossible to view some portions of the uterus. Some balloon catheters may avoid this problem by using balloons that may be inflated in the cervical canal. However, because the cervix tends to have a large number of nerve endings, which make the cervix sensitive to pressure from the inflated intra-cervical balloon, inflation of such balloons may cause considerable amounts of pain or discomfort.

Accordingly, it may be an improvement in the art to provide a balloon for a trans-cervical catheter used in uterine imaging that blocks less of the uterus during imaging. Similarly, it may be an improvement in the art to provide a balloon that causes less discomfort during its use.

### BRIEF SUMMARY OF THE INVENTION

This application relates generally to a trans-cervical balloon catheter. The catheter may have an elongated body for insertion into the uterus and a balloon located distally on the elongated body. The balloon has a substantially conical-shaped proximal end when inflated. The balloon may extend between a proximal origin and a distal origin. The balloon has a longitudinal midpoint located midway in between the proximal and distal origin. The balloon has a plane of maximum diameter at the widest part of the inflated balloon. The plane of maximum diameter is preferably located between the balloon's distal origin and the balloon's longitudinal midpoint.

The balloon may also taper from the plane of maximum diameter towards the proximal origin of the balloon in any manner that gives the proximal end of the balloon a conical-shaped appearance. For instance, the balloon may define a taper line that extends from the external surface of the elongated body at the proximal origin of the balloon and passes through the perimeter of the plane of maximum diameter. The angle between the taper line and the external surface of the elongated body may be between about 5 and about 60 degrees when the balloon is inflated. However, typically the taper angle of the inflated balloon is between about 20 and about 40 degrees. Moreover, in one embodiment, the taper angle may be between about 25 and 35 degrees.

Such a balloon may sit low in the internal uterine orifice and thereby block less of the uterus during imaging. Similarly, such a balloon may better follow the contours and natural shape of internal uterine orifice so as to create a better seal in the internal uterine orifice and cause less discomfort than some conventional balloon catheters.

### BRIEF DESCRIPTION OF THE DRAWINGS

The following description can be better understood in light of several Figures, in which:

FIG. 1 contains a perspective view of one embodiment of a catheter with a substantially conical-shaped balloon;

FIG. 2 contains a lateral cross-sectional view of one embodiment a catheter with a substantially conical-shaped balloon;

FIG. 3 contains a lateral cross-sectional view of the distal end of one embodiment of a catheter with a substantially conical-shaped balloon; and

FIG. 4 contains a lateral cross-sectional view of a catheter with a substantially conical-shaped balloon, where the balloon is anchored into a uterus.

Together with the following description, the Figures may help demonstrate and explain the principles of the invention and methods for making and using the invention. In the Figures, the thickness and configuration of components may be exaggerated for clarity. The same reference numerals in different Figures represent the same component.

### DETAILED DESCRIPTION OF THE INVENTION

The following description supplies specific details in order to provide a thorough understanding. Nevertheless, the skilled artisan would understand that the invention and associated methods of making and using the invention can be implemented and used without employing these specific details. Indeed, the invention and associated methods can be placed into practice by modifying the illustrated invention and associated methods and can be used in conjunction with any invention, system, component, and/or technique conventionally used in the art. For example, while the description below focuses on using the described balloon in conjunction with trans-cervical catheters, the balloon may also be implemented with many other types of catheters.

This application relates generally to a balloon with a substantially conical-shaped proximal end for use with a trans-cervical catheter. The described balloon may be used in conjunction with any type of trans-cervical catheter, including dual-lumen and single-lumen catheters. However, in order to better explain the implementation of the balloon with a substantially conical-shaped proximal end, this application describes the use of the balloon in the non-limiting embodiment of a trans-cervical, dual-lumen catheter.

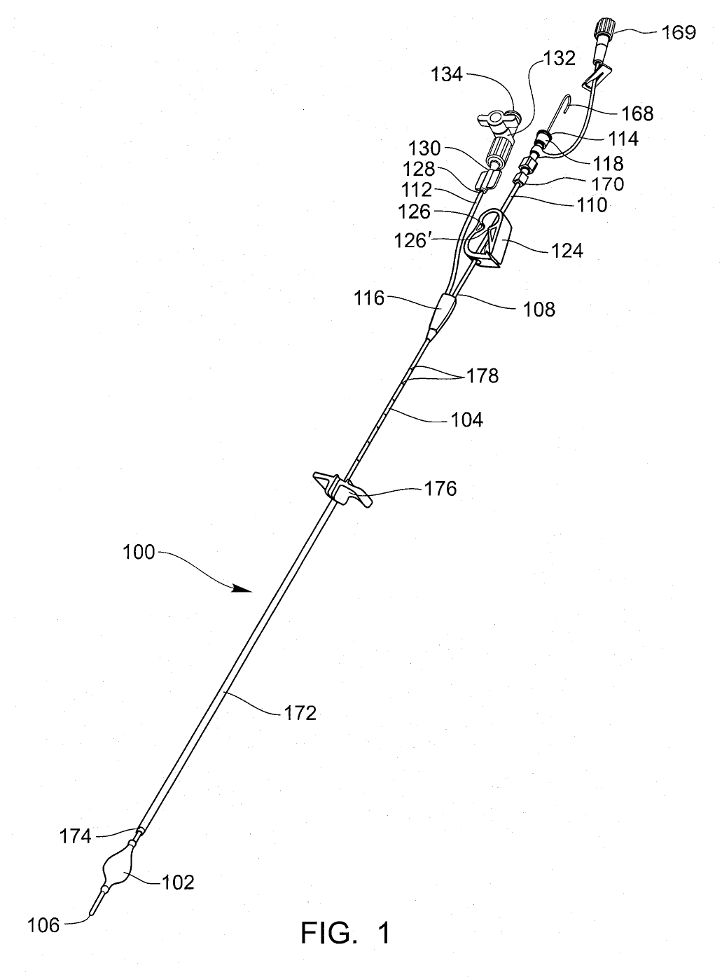

Although the described balloon may be used in conjunction with any known or novel trans-cervical, dual-lumen catheter and any conventional components, FIG. 1 illustrates a typical embodiment of a trans-cervical, dual-lumen catheter 100 with a balloon 102 having a substantially conical-shaped proximal end. Particularly, FIG. 1 depicts a dual-lumen catheter 100, a catheter with two lumens that may be used for entry into the uterine cavity of a female.

As depicted in FIG. 1, the catheter 100 may generally have an elongated flexible tubular catheter body 104 that extends from a distal end 106 to a proximal end 108. An intrauterine balloon 102 (depicted in the inflated state) may be disposed on the marginal distal end 106 of the body 104, and will be described in greater detail hereinafter. FIG. 1 also illustrates that the catheter 100 may include two fluid lines extending from the proximal end 108 of the body 104, where the two fluid lines may be a fluid line 110 and an inflation line 112, each of which are described below.

Of the two lines, the fluid line 110 is generally used to provide a communication path for introduction of a diagnostic fluid or material into the uterine cavity. Even though any fluid or material may be introduced into the uterine cavity through the fluid line 110, some examples of common fluids or materials may include a saline solution, an iodinated fluid, air, or other fluids known or used in the art.

The fluid line 110 may extend from its proximal end 114 through a fluid line coupler 116, enter or join the tubular body 104, and terminate in the distal end 106 of the catheter 100. Moreover, the fluid line 110 need not comprise a continuous tube made from a single material. Indeed, the fluid line 110 may be made in any manner known in the art. For example, the fluid line 110 may enter the tubular body 104 at the fluid line coupler 116, where the fluid line 110 may continue as a component formed in and with the tubular body 104. Also, as illustrated in FIG. 1, the proximal end 114 of the fluid line 110 may also have a conventional connector 118 for attaching various instruments or apparatus to the catheter. Any known or novel connector may be used for such a purpose, including a conventional Luer lock connector.

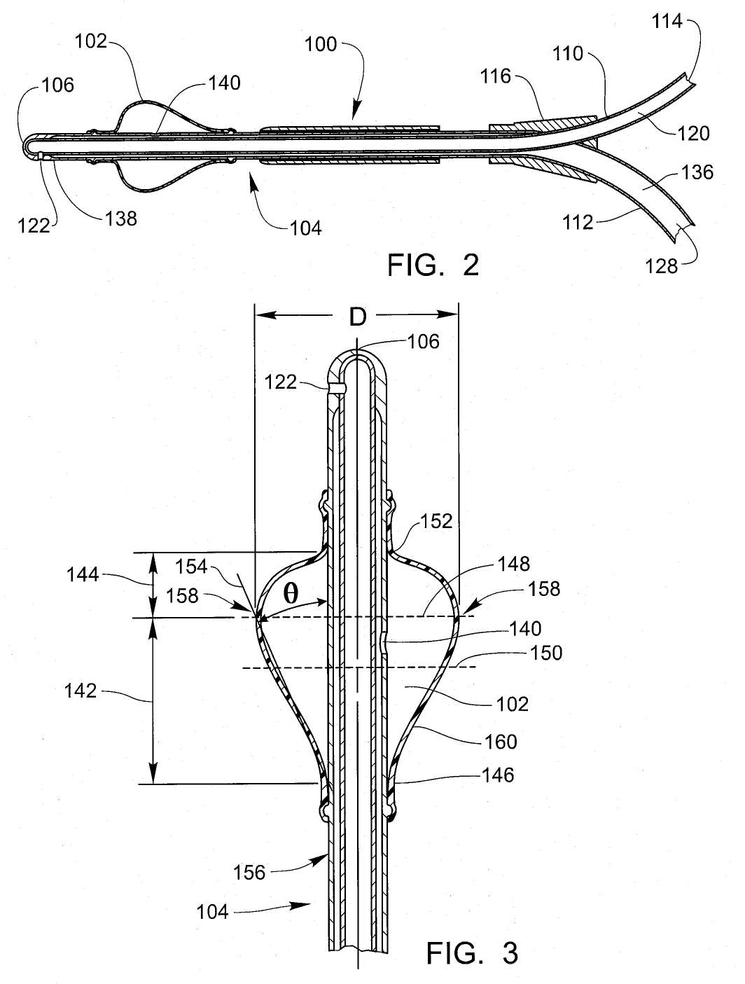

As depicted in FIG. 2, which shows a lateral cross-sectional view of one embodiment of a catheter 100, the fluid line 110 may define a working lumen 120 that may provide a communication path for the introduction of a diagnostic or other fluid in the uterine cavity. FIG. 2 illustrates that the working lumen 120 can start at the proximal end 114 of the fluid line 110 and may extend through the distal end 106 of the tubular body 104. Additionally, FIG. 2 illustrates that an aperture 122 located distally to the intrauterine balloon 102 may allow the working lumen 120 to communicate with the uterine cavity.

In some embodiments, the fluid line 110 may also have means for occluding the fluid line 110. For instance, FIG. 1 illustrates that a conventional plastic pinch clamp 124 may be slidably disposed on the fluid line 110 between the connector 118 and the fluid line coupler 116. The structure and operation of pinch clamps are well known in the art. Indeed, the pinch clamp 124 may occlude the fluid line 110 when the clamp 124 is squeezed into the “locked pinch mode.” When this occurs, opposing projections 126, 126′ on the pinch clamp 124 may operate on the fluid line 110 to occlude the working lumen 120. Such a pinch clamp 124 may be used for any desired purpose. For example, the pinch clamp 124 may be used to occlude the working lumen 120 after a diagnostic or other fluid has been introduced into the working lumen 120 but before the catheter 100 is inserted into the uterine cavity. In this manner, the pinch clamp 124 may serve to minimize the air injected into the uterine cavity. Additionally, the pinch clamp 124 may be used to occlude the fluid line 110 after a diagnostic or other fluid has been introduced into the uterus as well as throughout the imaging procedure.

As illustrated in FIG. 1, the second line extending from the proximal end 108 of the tubular body 104 may be the inflation line 112. The inflation line 112 may act as a communication path from an apparatus on the proximal end 128 of the line 112, to the balloon 102. FIG. 1 also illustrates that the proximal end 128 of the inflation line 112 may have a conventional connector 130 (e.g., a Luer lock connector), which may be connected to any desired apparatus. Indeed, FIG. 1 illustrates that the connector 130 may be removably connected to an inline rotary valve 132. In turn, FIG. 1 also illustrates that the rotary valve 132 may have a proximal end 134, which may be removably connected to any apparatus, including a conventional inflation syringe (not shown in the Figures).

FIG. 2 shows that the inflation line 112 may run from its proximal end 128 and pass through the fluid line coupler 116 to enter the tubular body 104. The inflation line 112 may then extend to the distal end 106 of the tubular body 104. FIG. 2 further illustrates that the inflation line 112 may define an inflation lumen 136 that may start at the proximal end 128 of the inflation line 112 and extend to the distal end 138 thereof. The inflation lumen 136 may communicate with the interior of the balloon 102 through an inflation aperture 140. In this manner, an inflation fluid, such as air, a saline solution, or any other desired material, may be used to inflate the intrauterine balloon 102.

Once the balloon 102 has been inflated, an apparatus, such as the earlier mentioned inline rotary valve 132 (as shown in FIG. 1), may be operated to maintain the balloon 102 in the inflated state. In operation, the balloon 102 may be inflated by an inflation device. An inflation syringe, for example, may inflate the balloon 102 by pushing the plunger into the body of the syringe. Once the balloon 102 is inflated, the inline rotary valve 132 may be rotated into the “closed” position, which may thereby prevent communication between the inflation syringe and the inflation lumen 136 and maintain the balloon 102 in its inflated state. When it is desirable to deflate the balloon 102, the inline rotary valve 132 may be turned to the “open” position, which may reestablish the communication between the syringe and the inflation lumen 136. In order to deflate the balloon 102, the plunger may be pulled within the body of the inflation syringe, as is known in the art.

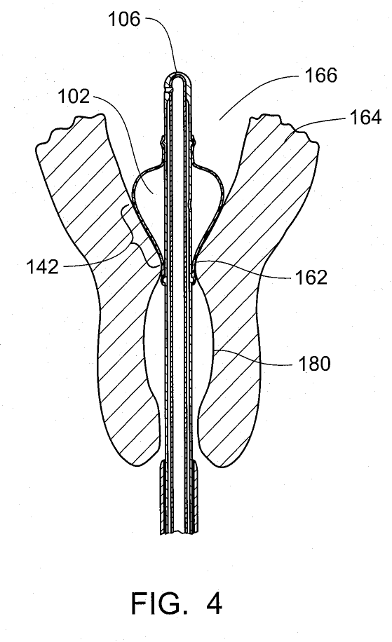

As mentioned earlier, the balloon 102 located on the marginal distal end 106 of the tubular body 104 may be substantially conically shaped when inflated. In particular, the proximal end 142 of the balloon 102 may have a substantially conical shape, while the distal end 144 of the balloon 102 may have any desired shape. FIG. 3 depicts that, in some embodiments, the proximal end 142 of the balloon 102 may extend from the balloon's proximal origin 146, or the point where the proximal end 142 of the inflated balloon 102 first contacts the exterior surface 156 of the elongated body 104, and then flare outward to the balloon's plane of maximum diameter 148. Although the proximal end 142 of the balloon may extend from the balloon's proximal origin 146 to any desired plane, in some embodiments, the proximal end 142 of the balloon 102 may only extend to the plane of maximum diameter 148. In some embodiments, the distal end 144 of the balloon 102 may extend from the plane of maximum diameter 148 to the balloon's distal origin 152, or the point where the distal end 144 of the inflated balloon first contacts the exterior surface 156 of the elongated body 104.

The balloon 102 may have any shape that allows the proximal end 142 to have a substantially conical-shaped appearance. Moreover, this appearance may be obtained in any manner. For example, FIG. 3 illustrates that, unlike spherical or ellipsoidal balloons that tend to have their plane of maximum diameter roughly coincide with their longitudinal midpoint (or the midpoint between the distal and proximal origin of the balloon), the plane of maximum diameter 148 in the described balloon 102 may be located distally to the described balloon's longitudinal midpoint 150. Indeed, the plane of maximum diameter 148 may be located anywhere between the longitudinal midpoint 150 of the balloon 102 and the balloon's distal origin 152. For example, the plane of maximum diameter 148 of the balloon 102 may be located roughly midway between the longitudinal midpoint 150 and the distal origin 152; the plane of maximum diameter 148 may be located closer to the distal origin 152 than the longitudinal midpoint 150; or the plane of maximum diameter 148 may be located closer to the longitudinal midpoint 150 than to the distal origin 152 of the balloon 102.

The described balloon 102 may taper from the plane of maximum diameter 148 towards the balloon's proximal origin 146 at any angle or in any desired manner. In other words, the balloon may flare outward from the proximal origin 146 to the plane of maximum diameter 148 at any desired angle or in any desired manner. For example, FIG. 3 depicts a taper line 154 drawn from the exterior surface 156 of the elongated body 104 at the proximal origin 146 of the balloon 102, to the perimeter 158 of the plane of maximum diameter 148. FIG. 3 illustrates that this taper line 154 may extend away from the exterior surface 156 of the elongated body 104 at the proximal origin 146 of the balloon 102 with any taper angle, where the taper angle is depicted by θ. In some embodiments, the taper line 154 may extend away from the exterior surface 156 at an angle θ between about 5 and about 60 degrees. In other embodiments, the angle θ between the taper line 154 and the exterior surface 156 may be between twenty and forty degrees. More specifically, in some embodiments, the angle θ between the taper line 154 and the exterior surface 156 may be between about twenty five and about thirty five degrees. For instance, the taper angle θ between the taper line 154 and the exterior surface 156 may be about thirty degrees when the balloon 102 is inflated to a typical volume, as will be described later.

Additionally, the wall 160 of the proximal end 142 of the balloon 102 may follow or vary from the taper line 154 in any manner that allows the balloon's proximal end 142 to be substantially conical shaped. For example, FIG. 3 depicts that the wall 160 of the proximal end 142 may extend from the exterior surface 156 at the proximal origin 146, curve so as to cross the taper line 154, and then bow back to the taper line 154 at the perimeter 158 of the plane of maximum diameter 148. However, as described in several examples that are not shown in the Figures, the wall of the proximal end of the balloon need not appear as the wall 160 in FIG. 3. For example, the wall of the proximal end of the balloon may substantially follow or overlap the taper line. However, in another example, the wall of the proximal end may curve from the taper line so that as the wall extends from the proximal origin, the wall may bow in towards the exterior surface, and then extend out to the perimeter of the plane of maximum diameter 148 where the wall contacts the taper line. In yet another example, the wall may curve from the taper line 154 so that the wall extends from the proximal origin 146, bow out away from the exterior surface 156 so as to cross the taper line 154 near the proximal origin 146, and then extend back to the plane of maximum diameter 148 where the wall 160 crosses the taper line 154 again.

Thus, as previously described, the substantially conical-shaped proximal end 142 of the balloon 102 need not be perfectly conical. Indeed, the proximal end 142 may be any shape that gives the proximal end 142 a substantially conical-shaped appearance. In some non-limiting examples of possible shapes of the proximal end 142, the proximal end 142 may be funnel-like, elliptical cone-like, rain-drop-like, or tear-drop-like and still be considered as being substantially conically shaped.

As mentioned, the distal end 144 of the balloon 102 may have any desired shape. For example, the distal end 144 of the balloon 102 may be semi-spherical, semi-elliptical, substantially planar, or any desired or arbitrary shape. For example, FIG. 3 shows that the distal end 144 of the balloon 102 may be substantially semi-spherical in shape. In that Figure, the semi-spherical distal end 144 and the substantially conical-shaped proximal end 142 may give the balloon 102 a raindrop-like appearance. However, in another example (not shown in the Figures), the distal end of the balloon may be substantially planar. In this example, the substantially planar distal end of the balloon and the substantially conical proximal end may give the balloon a cone or funnel-like appearance. Such an embodiment may be advantageous because it may reduce the amount of the balloon that extends into the uterine cavity. In this manner, the balloon from this example may further increase the amount of the uterus that can be visualized during imaging, while still sealing the uterus.

The described balloon may be any size suitable for insertion and placement in the uterus. In one embodiment, the balloon 102 may have a diameter D at the plane of maximum diameter 148 between about 4 and about 15 millimeters when the balloon is inflated. In a typical embodiment, the balloon 102 may have a maximum diameter D at the plane of maximum diameter between about 6 and about 8 millimeters when the balloon is inflated. Indeed, in one embodiment the maximum diameter at the plane of maximum diameter may be about 7 millimeters.

Additionally, the length of the balloon 102, or the distance between the proximal origin 146 and the distal origin 152, may be any length suitable for insertion and placement in the uterus. For instance, the balloon's length while deflated may be between about 8 and about 18 millimeters. This length may vary little, if at all, when the balloon is inflated. In one example of a typical balloon length, the balloon may be about 11 millimeters long.

Furthermore, the balloon 102 may have any desired volume when inflated. For instance, the balloon 102 may be designed to be inflated so as to have a volume between about 0.03 and about 1.5 cubic centimeters. However, in a typical embodiment, the balloon 102 may be designed to have a volume between about 0.1 and about 0.5 cubic centimeters. Indeed, in one embodiment, the balloon 102 may have a volume of about 0.3 cubic centimeters; although the balloon 102 volume may be more or less than that depending on the elasticity of the balloon material, which is discussed hereinafter.

The substantially conical-shaped proximal end 142 of the balloon 102 may offer several advantages. For instance, FIG. 4 illustrates that the substantially-conical shape of the proximal end 142 of the balloon 102 may allow the balloon 102 to follow the contours and natural shape of the internal uterine orifice 162 better than some balloons currently used. Accordingly, the balloon 102 may be more comfortable than some conventional balloons. Furthermore, the shape of the described balloon 102 may also allow the intrauterine balloon 102 to sit low in the internal uterine orifice 162. In this manner, the described balloon 102 may tend to block less of the uterus 164 during imaging and, therefore, the balloon 102 may allow a more thorough examination of the uterus 164. Additionally, FIG. 4 illustrates that the shape of the described balloon 102 may allow a significant amount of the proximal end 142 of the balloon 102 to come in contact with the surface of the internal uterine orifice 162. Because a larger amount of the described balloon 102 may come in contact with the surface of the internal orifice 162 than conventional balloons, the described balloon 102 may seal the intrauterine cavity 166 without requiring the balloon 102 to exert as much pressure on the internal orifice 162 as some conventional balloons. For this reason, the described balloon 102 may also decrease the discomfort associated with the use of conventional trans-cervical catheters.

In addition to the previously mentioned components of the balloon and described catheter, any component, apparatus, or system may be used in conjunction with the described balloon and/or catheter. For instance, the trans-cervical catheter and balloon may be used along with a stylet and a cylindrical collar member.

In some embodiments, the fluid line 110 may be used along with a removable stylet 168, as illustrated in FIG. 1. The stylet 168 may be disposed in the fluid line 110 and extend through the working lumen 120 in the tubular body 104. Among other functions known in the art, the stylet 168 may be employed to prevent the catheter 100 from bending and flexing excessively in the vaginal canal, especially in cases where insertion of the catheter 100 into the cervical canal is difficult due to stenosis or some other condition. In such circumstances, the stylet 168 may be disposed in the catheter 100 before the catheter 100 is positioned in the uterus. After positioning, or at any desired time, the stylet 168 may be removed to allow any apparatus to be connected to the fluid line 110. For example, it may be desirable to connect an apparatus at connector 169, which may be a conventional Luer connector. Although the stylet 168 in FIG. 1 is depicted as having a crooked-shaped proximal end, any type of stylet may be used with the catheter 100. For example, a stylet may have a connector, such as a Luer lock, on its proximal end. In this example, the stylet may be easily secured to the catheter 100 as well as easily removed. In embodiments that do not include the stylet 168, apparatus may be connected to fluid line 110 at connector 170, which may also be a conventional Luer connector.

In some embodiments, such as the embodiment of FIG. 1, the flexible tubular body 104 may have a cylindrical collar member 172 that is slidably mounted on the body 104 between the balloon 102 and the fluid line coupler 116. Such a cylindrical collar member 172 may serve any purpose. For example, the cylindrical collar member 172 may be used to add rigidity to the flexible tubular body 104 so as to facilitate placement of the catheter 100.

Any cylindrical collar member 172 may be used with the trans-cervical catheter 100. For example, a cylindrical collar member 172 may include a cut, perforation, tearable seam, or longitudinal weakening, so that the cylindrical collar member 172 can be removed or peeled away from the catheter 100 after catheter placement and prior to imaging. In another example, the distal end 174 of the cylindrical collar member 172 may have an outwardly extending circumferential flange (not shown in Figures), which may help prevent inserting the collar member 172 into the cervical canal. However, in other embodiments, the distal end 174 of the cylindrical collar member 172 may be tapered, as is illustrated in FIG. 1.

The proximal end 174 of the cylindrical collar member 172 may have a cylindrical collar member grip 176, a non-limiting example of which is shown in FIG. 1. Such a collar grip 176 may serve any desired purpose. For example, such a grip 176 may provide a surface to be grasped and used to manipulate and direct movement of the catheter 100. In another example, such a grip may be used to break and peel away the cylindrical collar member 172. Additionally, in some embodiments, the cylindrical collar member 172 may also have a large enough diameter to allow the collar member 172 to slide over a deflated and collapsed balloon (not illustrated in the Figures).

In some embodiments, the tubular body 104 of the catheter 100 may also include any form of marking(s) to help indicate catheter 100 insertion depth and thereby be used to avoid uterine perforation. Additionally, the markings may be located on the tubular body 104 in any desired manner or configuration. For example, markings on the tubular body 104 of the catheter 100 used without a cylindrical collar member may be located distal on the tubular body but proximal to the balloon. However, as illustrated in FIG. 1, in another example, a catheter 100 that is used in conjunction with a cylindrical collar member 172 may have markings 178 located towards the proximal end 108 of the tubular body 104. In this example, some of the markings 178 may be covered by the cylindrical collar member 172 as the tubular body 104 is pushed through the collar member 172. Thus, insertion depth may be measured by reference to the uncovered markings 178.

Each of the aforementioned components of the catheter may be made from any materials suitable for use in a trans-cervical catheter. For instance, the components may be made from medical-grade nylon, polyethylene, polyurethane, polyvinyl chloride, mixtures thereof, silicone, latex, polypropylene, neoprene, or a composite. The balloon 102, for example, may be made from polyurethane or any other medical-grade elastomeric material. In one embodiment, the balloon may have a Shore durometer measurement between about 70 and 95 on the A scale. Moreover, the wall 160 thickness of the balloon 102 may have any desired thickness. For example, the wall 160 thickness may range between 0.0005 and 0.1 inches.

The balloon 102 can be made using any known method or technique. For example, the balloon 102 may be molded or extruded as known in the art. The entire balloon 102 may be made of a single piece of elastomeric material or the balloon 102 may be made of two or more pieces of material. In one example, a proximal end 142 of the balloon 102 may be made of a substantially conical-shaped piece of elastomeric material that is, in turn, fused to a distal end 144 of the balloon 102 made from a substantially semi-spherical piece of elastomeric material. Additionally, the balloon 102 may be attached to the tubular body 104, and pieces of the balloon 102 may be attached to each other, through any known or novel method or technique. For instance, the balloon 102 may be attached to the tubular body 104 through adhesive bonding, heat sealing, and/or mechanically clamping.

A catheter 100 with a balloon 102 having a substantially conical-shaped proximal end 146 may be used in any manner or method. Indeed methods for using catheters with intrauterine balloons are well known in the art. Nonetheless, in order to better explain the described balloon 102 and its use, a non-limiting example of the balloon's use in conjunction with a dual-lumen catheter is given herein.

A diagnostic fluid may be introduced by an apparatus, located at the proximal end 114 of the fluid line 110, into the working lumen 120 of the catheter 100. Once the working lumen 120 is substantially filled with the fluid, the fluid line 110 may be occluded with a pinch clamp 124, as previously described. A portion of the distal end 106 of the tubular body 104, including the balloon 102, may be inserted in and through the cervix and then into uterine cavity 166.

After insertion into the uterine cavity 166, an apparatus, such as an inflation syringe, located on the proximal end 128 of the inflation line 112 may be used to introduce air or fluid through the inflation lumen 136 and into the interior of the balloon 102. In this manner, the balloon 102 may be inflated. The balloon 102 may be progressively inflated so that the balloon 102 may be of different sizes and variations of the balloon's substantially conical-shaped proximal end 142. Accordingly, the balloon 102 may be inflated sufficient to seal the internal uterine orifice 162 without causing unnecessary discomfort from excessive pressure. For instance, after the catheter 100 has been inserted into the uterus and the balloon 102 has been inflated, more air, saline solution, etc. may be added to the balloon 102 if diagnostic fluid begins to leak from the uterine cavity 166.

When the balloon 102 is inflated as desired, the inflation line 112 may be occluded through the use of an apparatus, such as the previous mentioned inline rotary valve 132. The catheter 100 may then be extracted slightly, so as to bring the substantially conical-shaped proximal end 142 of the balloon 102 in contact with the internal orifice 162 of the uterus 164. In this way, the balloon 102 may seal the uterine cavity 166.

Once the cavity 166 has been sealed, the pinch clamp 124 on the fluid line 110 may be removed from the “locked pinch mode” and the diagnostic fluid may be introduced into the uterine cavity 166 so as to expand the cavity 166 for imaging. The filled cavity 166 may then be imaged through any method known in the art, including TVUS and HSG. After completion of imaging, the inline rotary valve 132 may be opened and the inflation syringe may be used to deflate the balloon 102. When the balloon 102 is deflated the tubular body 104 may be withdrawn from the uterine cavity 166 and cervical canal 180.

In addition to any previously indicated modification, numerous other variations and alternative arrangements may be devised by those skilled in the art without departing from the spirit and scope of the invention, and appended claims are intended to cover such modifications and arrangements. Thus, while the invention has been described above with particularity and detail in connection with what is presently deemed to be the most practical and preferred aspects of the invention, it will be apparent to those of ordinary skill in the art that numerous modifications, including, but not limited to, form, function, manner of operation, manufacture, and use may be made without departing from the principles and concepts set forth herein. Also, as used herein, examples and embodiments are meant to be illustrative only and should not be construed as limiting in any manner.

1. A trans-cervical balloon catheter for entry into a uterus, comprising:
an elongated tubular catheter body for insertion into the uterus, wherein the tubular body comprises a distal end and a proximal end; and
an inflatable balloon disposed at a marginal distal end the catheter body, wherein the inflatable balloon comprises a distal end of the balloon and a proximal end of the balloon, and wherein the proximal end of the balloon is substantially conically shaped when inflated.

2. The trans-cervical balloon catheter of claim 1, wherein the balloon further comprises a distal origin, a longitudinal midpoint, and a plane of maximum diameter, and wherein the plane of maximum diameter is located between the distal origin and the longitudinal midpoint.

3. The trans-cervical balloon catheter of claim 1, wherein the balloon comprises a volume between about 0.03 and about 1.5 cubic centimeters when inflated.

4. The trans-cervical balloon catheter of claim 1, wherein the balloon comprises a volume of between about 0.1 and about 0.5 cubic centimeters when inflated.

5. The trans-cervical balloon catheter of claim 1, wherein the balloon at the plane of maximum diameter comprises a diameter between about 4 and about 15 millimeters.

6. The trans-cervical balloon catheter of claim 1, wherein the balloon at the plane of maximum diameter comprises a diameter between about 6 and about 8 millimeters.

7. The trans-cervical balloon catheter of claim 1, wherein the balloon has a taper angle between an exterior surface of the elongated tubular body and a taper line, which extends from a proximal origin of the balloon to a perimeter of the plane of maximum diameter, and wherein the taper angle is between about 5 and about 60 degrees when the balloon is inflated.

8. The trans-cervical balloon catheter of claim 1, wherein the balloon has a taper angle between an exterior surface of the elongated tubular body and a taper line, which extends from a proximal origin of the balloon to a perimeter of the plane of maximum diameter, and wherein the taper angle is between about 20 and about 40 degrees when the balloon is inflated.

9. The trans-cervical balloon catheter of claim 1, wherein the balloon has a taper angle between an exterior surface of the elongated tubular body and a taper line, which extends from a proximal origin of the balloon to a perimeter of the plane of maximum diameter, and wherein the taper angle is between about 25 and about 35 degrees when the balloon is inflated.

10. The trans-cervical balloon catheter of claim 1, wherein the tubular body comprises two lumens.

11. The trans-cervical balloon catheter of claim 1, wherein the tubular body comprises a fluid line and an inflation line.

12. A trans-cervical balloon catheter for entry into a uterus, comprising:
an elongated tubular catheter body for insertion into the uterus, wherein the tubular body comprises a distal end and a proximal end; and
an inflatable balloon disposed on the marginal distal end the catheter body, the balloon comprising:
a distal end of the balloon;
a proximal end of the balloon, wherein the proximal end of the balloon is substantially conically shaped when inflated;
a distal origin;
a longitudinal midpoint; and
a plane of maximum diameter, wherein the plane of maximum diameter is located between the distal origin and the longitudinal midpoint.

13. The trans-cervical balloon catheter of claim 12, wherein the balloon has a taper angle between an exterior surface of the elongated tubular body and a taper line, which extends from an exterior surface of the catheter body at a proximal origin of the balloon to a perimeter of the plane of maximum diameter, and wherein the taper angle is between about 20 and about 40 degrees when inflated.

14. The trans-cervical balloon catheter of claim 12, wherein the balloon has a taper angle between an exterior surface of the elongated tubular body and a taper line, which extends from an exterior surface of the catheter body at a proximal origin of the balloon to a perimeter of the plane of maximum diameter, and wherein the taper angle is between about 25 and about 35 degrees when inflated.

15. The trans-cervical balloon catheter of claim 13, wherein the balloon comprises a diameter at the plane of maximum diameter between about 4 and about 15 millimeters.

16. The trans-cervical balloon catheter of claim 14, wherein the balloon comprises a diameter at the plane of maximum diameter between about 6 and about 8 millimeters.

17. The trans-cervical balloon catheter of claim 12, wherein the tubular body comprises two lumens.

18. The trans-cervical balloon catheter of claim 17, wherein the two lumens comprise a fluid line and an inflation line.

19. The trans-cervical balloon catheter of claim 12, wherein the balloon comprises a volume between about 0.1 and about 0.5 cubic centimeters when inflated.

20. A trans-cervical balloon catheter for entry into a uterus, comprising:
an elongated tubular catheter body for insertion into the uterus, wherein the tubular body comprises a distal end, a proximal end, a fluid line, and an inflation line; and
an inflatable balloon disposed on the marginal distal end the catheter body, the balloon comprising:
a distal end of the balloon;
a proximal end of the balloon, wherein the proximal end of the balloon is substantially conically shaped when inflated;
a distal origin;
a proximal origin;
a longitudinal midpoint between the distal origin and the proximal origin; and
a plane of maximum diameter, wherein the plane of maximum diameter is located between the distal origin and the longitudinal midpoint; and
wherein the balloon has a taper angle between an exterior surface of the elongated tubular catheter body and a taper line, which extends from the exterior surface at a proximal origin of the balloon to a perimeter of the plane of maximum diameter, and wherein the taper angle is between about 20 and about 40 degrees when inflated.
